(19) United States
(12) Patent Application Publication
(10) Pub. No.: US 2007/0225238 A1
(43) Pub. Date: Sep. 27, 2007

(54) Inhibitors of carbonyl reductase for treatment using anthracyclines

(76) Inventor: Henry A. Charlier JR., Boise, ID (US)

Correspondence Address: PEDERSEN & COMPANY, PLLC, P.O. BOX 2666, BOISE, ID, 83701, US

(21) Appl. No.: 11/711,490

(22) Filed: Feb. 26, 2007

Related U.S. Application Data
(60) Provisional application No. 60/776,269, filed on Feb. 24, 2006.

(51) Int. Cl. A61K 31/704; A61K 31/095

(52) U.S. Cl. 514/34; 514/706

(57) ABSTRACT

Compositions of matter and methods of using the compositions of matter are disclosed for preventing or reducing cardiotoxicity during or after cancer treatment with anthracycline drugs, and preventing or reducing resistance to anthracycline drugs, both of which are believed to be caused by human enzyme carbonyl reductase. Thus, the compositions and methods may be used to reduce the dosages of anthracycline anti-cancer drugs necessary to produce a desired cancer-cell-killing performance in a cancer patient. The compositions of matter and methods comprise inhibiting enzyme(s) that catalyze formation of metabolites that build up during or after treatment with anthracycline cancer drugs, said metabolites being ones that are believed to disrupt heart muscle processes and therefore to interfere with heart function. Preferred embodiments comprise treating cancer patients with a pharmaceutical composition comprising 2,2′-thio-bis(4,6-dichlorophenol) (also called “bithionol” or “bis(2-hydroxy-3,5-dichlorophenyl)sulfide”) and/or 2,2′-sulfinyl-bis(4,6-dichlorophenol) (also called “bithionol sulfoxide”) and/or derivatives or analogs thereof.

This application claims priority of Provisional Application No. 60/776,269, filed Feb. 24, 2006, and entitled “Inhibitors Of Carbonyl Reductase For Treatment Using Anthracyclines”, which is hereby incorporated by reference.

Some activities related to this invention were conducted with support by the United States National Institute of Health, #NIH/P20RR16454.

### BACKGROUND OF THE INVENTION

1. Field of the Invention

This invention relates to compositions of matter and methods of using said compositions for inhibiting human reductase enzyme(s) that destroy the cell-killing efficacy of anthracycline cancer drugs, and that catalyze the formation of dangerous or damaging metabolites during or after cancer treatment. More specifically, embodiments of the invented compositions and methods of using said compositions inhibit human carbonyl reductase, thus inhibiting conversion of anthracycline to metabolites that are less effective cell-killing agents and that also lead to cardiotoxicity during or after treatment of cancer patients. Thus, the invented compositions and methods are believed to reduce the amount needed, and the cardiotoxic side-effects, of anthracyclines in cancer treatment.

2. Related Art

Anthracyclines are a family of drugs that are effective anti-neoplastic agents, and are commonly used to treat cancer, including leukemia, soft tissue sarcomas, and breast and lung cancer. Anthracyclines intercalate into DNA and are described as topoisomerase Type II poisons. The anthracycline family comprises adriamycin, daunomycin, daunorubicin, doxorubicin, epirubicin, and idarubicin. See, for example, the representations of doxorubicin and daunorubicin shown in FIG. 1.

While the anthracyclines are known to be potent anti-tumor drugs, their use has been limited due to potentially life-threatening cardiotoxicity associated therewith. This problem may be described as cumulative dose-dependent cardiotoxicity, which can ultimately result in congestive heart failure. There is significant evidence that the toxic effects on the heart associated with anthracycline-based cancer treatment are largely attributable to anthracycline alcohol metabolite(s) that form and accumulate in cardiac cells. These metabolites are known to disrupt several key processes in heart muscle and thus impair heart function. See, for example, Minotti, et al., “Anthracyclines: Molecular Advances and Pharmacologic Developments in Antitumor Activity and Cardiotoxicity,” Pharmacological Reviews, 56: 185-229, 2004.

Enzymes belonging to the aldo-keto reductase and short chain dehydrogenase/reductase protein superfamilies catalyze the formation of the anthracycline metabolites. Of these enzymes, carbonyl reductase (“CR”) has been specifically linked to the development of anthracycline-induced cardiotoxicity. See, for example, Olson, et al., “Protection from Doxorubicin-Induced Cardiac Toxicity in Mice with a Null Allele of Carbonyl Reductase 1,” Cancer Research, 63, 6602-6606, Oct. 15, 2003. Findings that support the hypothesis that CR is a key factor in anthracycline-induced cardiotoxicity include studies wherein heart-specific over-expression of human carbonyl reductase in transgenic mice substantially increased the development of cardiotoxicity after anthracycline treatment. See, for example, Forrest, et al., “Human Carbonyl Reductase Overexpression in the Heart Advances the Development of Doxorubicin-induced Cardiotoxicity in transgenic Mice,” Cancer Research, 60, 5158-5164, Sep. 15, 2000.

Further, several studies have implicated the reduction of anthracyclines by carbonyl reductase in drug resistance. This is largely because the alcohol metabolites of anthracyclines have been shown to exhibit significantly reduced anticancer properties. Relevant to this are studies performed by Tanaka, et al. (reported in Tanaka, et al., “An Unbiased Cell Morphology-Based Screen for New, Biologically Active Small Molecules,” PLoS Biology, volume 3, issue 5, 0764-0776, May 2005). Tanaka, et al. report that a potent inhibitor of human carbonyl reductase (3-(7-isopropyl-4-(methylamino)-7H-pyrrolo[2,3-d]pyrimidin-5-yl)phenol, also known as hydroxyl-PP-me), when co-administered with daunorubicin to A549 adenocarcinoma cells, was found to enhance the cytotoxicity of daunorubicin. It was concluded that inhibition of carbonyl reductase led to enhanced cytotoxicity of daunorubicin.

FIG. 2 illustrates carbonyl reductase catalysis (reduction via NADPH+H⁺ mechanism) of the anthracycline daunorubicin to daunorubicinol. While daunorubicin is an effective anti-cancer agent by means of its effective disruption of DNA replication, daunorubicinol exhibits reduced anti-cancer properties and is a potent cardiotoxin. Therefore, conversion of the anthracycline to the alcohol metabolite not only creates a potent cardiotoxin, but also lowers the efficacy of the treatment for a given amount of anthracycline.

Therefore, the inventor believes that there is a need for pharmaceutical interventions that block the action of human carbonyl reductase. The inventor believes that such pharmaceutical interventions will increase the efficacy of anthracycline therapy in cancer/tumor treatment by preventing or reducing conversion of anthracyclines to less potent cell-killing species and by reducing the risk of cardiotoxicity.

### SUMMARY OF THE INVENTION

The present invention comprises compositions of matter, and methods of treating patients with the compositions of matter, to prevent or reduce conversion in the human body of anthracycline drugs to metabolites that are less effective for cancer treatment and that are also believed to produce cardiotoxicity during or after cancer treatment. Hence, by using embodiments of the invention, the effectiveness of a given dose of anthracycline drugs may increase and the cardiotoxicity typically associated with said treatment may lessen.

Embodiments of the invention comprise inhibiting carbonyl reductase enzyme(s), and/or other enzyme(s) that catalyze anthracycline conversion to anthracycline metabolites. This inhibition has the direct effect of maintaining concentrations of anthracyclines, which are desirable for their cell-killing abilities, and, hence, for their cancer-cell-killing abilities. This inhibition also has the indirect effect of reducing formation of metabolites that build up during or after treatment with anthracycline cancer drugs, said metabolites being ones that are believed to disrupt heart muscle processes and therefore interfere with heart function. Therefore, much less anthracycline drug is expected to be needed to achieve the desired killing of cells, and much less cardiotoxic metabolite will be produced over the duration of the cancer treatment.

Preferred embodiments of the invention comprise treating cancer patients with a pharmaceutical composition comprising 2,2′-thio-bis(4,6-dichlorophenol) (also called “bithionol”) or “bis(2-hydroxy-3,5-dichlorophenyl)sulfide”) and/or 2,2′-sulfinyl-bis(4,6-dichlorophenol) (also called “bithionol sulfoxide”) and/or derivatives or analogs thereof. The preferred composition of bithionol, bithionol sulfoxide, and/or derivatives or analogs thereof, may be administered to a human (or other mammal) in a pharmaceutical composition also comprising at least one anthracycline compound, or may be administered separately from the at least one anthracycline compound either at the same as the anthracycline(s), or any different time found to be effective for inhibiting formation of the anthracycline metabolites.

Therefore, an object of the present invention is to inhibit one or more of the members of the aldo-keto reductase and/or short chain dehydrogenase/reductase protein superfamilies, which catalyze the conversion of anthracyclines to anthracycline metabolites. The preferred compositions and methods are adapted to inhibit member(s) of these superfamilies that is/are currently associated with cardiotoxicity from anthracycline chemotherapy, that is, human carbonyl reductase. A synergistic effect of inhibiting said reductase enzyme is expected to be that lower dosages of the anthracycline drug will be effective for cancer-cell-killing.

### BRIEF DESCRIPTION OF THE DRAWINGS

FIG. 1 is a representation of an anthracycline compound, which may be doxorubicin when R═OH, or daunorubicin when R═H.

FIG. 2 is a representation of a carbonyl reductase —NADPH mechanism for reducing the anthracycline daunorubicin to the anthracycline alcohol metabolite daunorubicinol.

FIG. 3A shows the chemical structure of 2,2′-thio-bis(4,6-dichlorophenol) (also called “bithionol” or “bis(2-hydroxy-3,5-dichlorophenyl)sulfide”).

FIG. 3B shows the chemical structure of 2,2′-sulfinyl-bis(4,6-dichlorophenol) (also called “bithionol sulfoxide”).

FIGS. 4A, B, C and D are four compounds tested for inhibition of the enzymes of interest, and which showed no inhibition.

FIGS. 5A, B and C are three compounds tested for inhibition of the enzymes of interest, and which showed inhibition.

FIGS. 6A and B are graphs showing bithionol sulfoxide as noncompetitive inhibitor against both menadione (varying NADPH, FIG. 6A) and NADPH (varying menadione, FIG. 6B).

FIG. 6C illustrates the structure of menadione, and FIG. 6D illustrates the structure of 4-Benzoylpyridine.

FIG. 7 describes inhibition patterns seen with bithionol and bithionol sulfoxide, wherein NC stands for “non-competitive” and C stands for “competitive.”

FIGS. 8 and 9 portray results from intrinsic protein fluorescence quenching experiments, which confirm that both bithionol and bithionol sulfoxide bind to both free enzyme and enzyme NADP⁺ binary complex, and that E-NADP⁺ is bound by bithionol sulfoxide.

FIGS. 10A and 10B summarize data showing that both bithionol (FIG. 10A) and bithionol sulfoxide (FIG. 10B) bind to multiple enzyme forms, as determined from inhibition and protein fluorescence quenching studies such as those represented in FIGS. 6A, 6B, and 7-9.

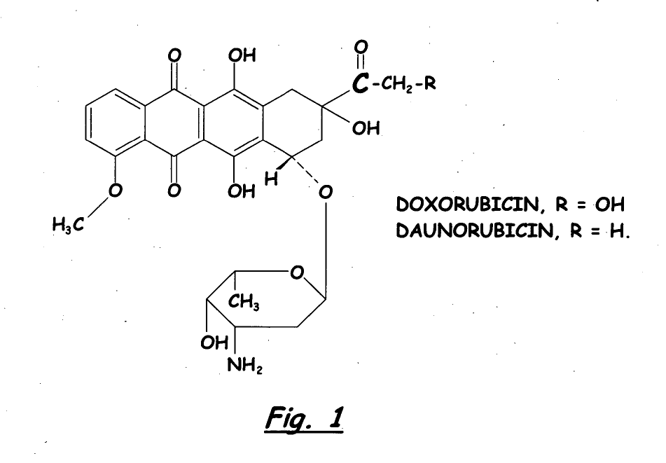

### DESCRIPTION OF THE PREFERRED EMBODIMENTS

Referring to the figures, there are illustrated several, but not the only, embodiments, and text results from embodiments, of the invented composition of matter and methods for enhancing the efficacy of anthracycline drug cancer treatment and/or reducing side-effects thereof. The preferred methods and compositions of matter may maintain effective concentrations of anthracycline(s) during cancer treatment, by preventing or reducing conversion of the anthracycline(s) to metabolites that are less effective or ineffective as cancer-cell-killing species. The preferred methods and compositions also prevent or reduce the potentially life-threatening cardiotoxicity associated with anthracycline chemotherapy for cancer patients.

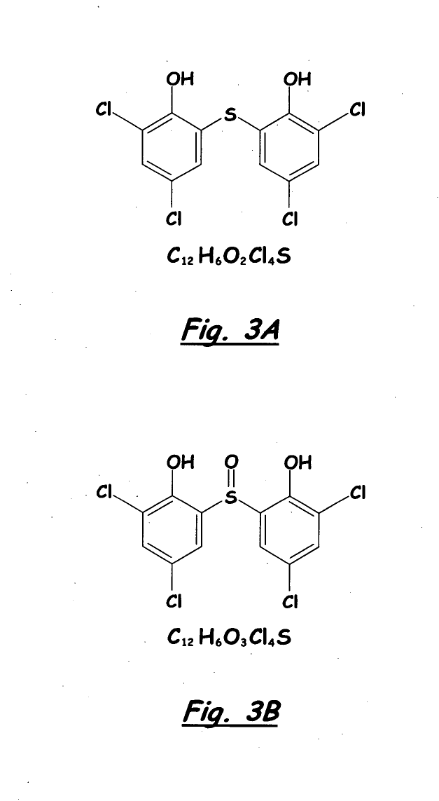

The preferred compositions of matter comprise one or both of 2,2′-thio-bis(4,6-dichlorophenol) (also called “bithionol” or “bis(2-hydroxy-3,5-dichlorophenyl)sulfide”) and 2,2′-sulfinyl-bis(4,6-dichlorophenol) (also called “bithionol sulfoxide”). See FIGS. 3A and 3B. Synthesis of each of these compounds is known, and these compounds are commercially available, for example, from Sigma Aldrich (T9881 bithionol and S8259 bithionol sulfoxide). Also, it is expected that derivatives or analogs of these compounds may be effective in the place of one or both of these two compounds, or as a supplement to one or both of these compounds.

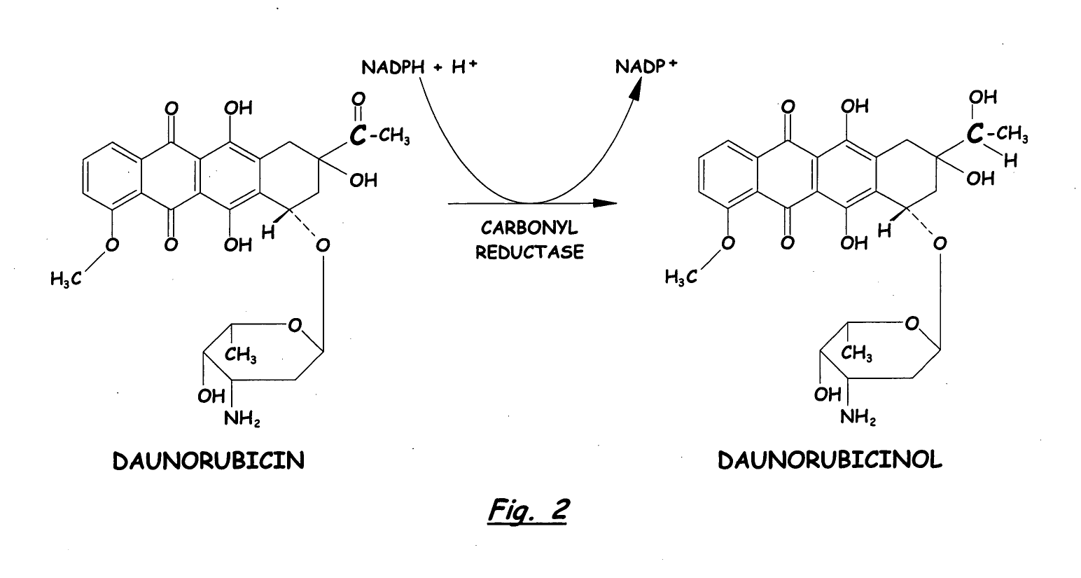

The disease or condition of cardiotoxicity related to anthracycline drugs is briefly described above in the Related Art Section. Multiple studies point to human carbonyl reductase (such as carbonyl reductase 1) having a role in the production of the anthracycline metabolites believed to cause cardiotoxity side effects in cancer patients either near the time of the chemotherapy or at some later time. See FIG. 2 for one example of a mechanism of carbonyl reductase reduction of an anti-cancer anthracycline to a cardiotoxic alcohol metabolite. C13-hydroxy-metabolites are believed to be the principle cardiotoxic agents resulting from enzyme action upon anthracyclines. Human “carbonyl reductase” is believed to comprise several isoenzymes, which are members of the short-chain dehydrogenase/reductase superfamily and monomeric or tetrameric with subunit molecular weight of approximately 30 kDa. Carbonyl reductase uses NADPH, and may have physiological roles including quinone detoxification or other roles.

In addition, as the anthracycline metabolite(s) are believed to not possess the anti-neoplastic properties of the parent anthracycline(s), carbonyl reductase may also contribute to anthracycline drug resistance. In other words, carbonyl reductase may reduce anthracycline concentrations in the human body by catalyzing conversion of the anthracycline, and, hence, may reduce the amount of cancer cells killed by a given dose of anthracycline drug.

The preferred compositions and methods, comprising 2,2′-thio-bis(4,6-dichlorophenol) (called hereafter “bithionol”) and 2,2′-sulfinyl-bis(4,6-dichlorophenol) (called hereafter “bithionol sulfoxide”), have been shown by the inventor to inhibit carbonyl reductase, and are envisioned to allow an increase in anthracycline chemotherapy by offsetting the negative side effects of this chemotherapy. Also, as discussed above, the preferred compositions and methods may decrease anthracycline drug resistance, further improving the results of anthracycline chemotherapy.

The inventor believes that embodiments of the invention may comprise taking bithionol and/or bithionol sulfoxide orally, which has advantages for most patients and their care-givers. Bithionol has been used in the past as an anti-microbial compound, and, in internal medicine, for treating liver fluke infections, but, to the inventor's knowledge, neither bithionol nor bithionol sulfoxide has previously been used in any process for improving efficacy of drugs used in cancer treatment or for treating or preventing side effects of cancer treatment or cardiotoxicity. However, the inventor believes that previously-used clinical doses of bithionol for liver fluke treatment may provide a starting place for finding safe doses for cancer treatment enhancement and cardiotoxicity prevention/treatment. For example, oral doses (given every other day for 10-15 doses) in the range of 30-50 mg of bithionol per kilogram of human body weight have been used to treat liver flukes, and this may be a starting place for determining a safe dose for embodiments of this invention. See Bacq, et al., “Successful Treatment of Acute Fascioliasis with Bithionol,” Hepatology, 1991; 14(6); 1066-9 (ISSN: 0270-9139). While the above-reported dose has been effective and considered safe in the context of liver fluke treatment, the dose of bithionol and/or bithionol sulfoxide may be increased in the context of the invented cancer-treatment methods, if the side-effects of the higher doses are relatively benign compared to the benefits of the increased anthracycline-based cancer-treatment efficacy (lowering of anthracycline dose and/or lowering of cardiotoxicity) that may be achievable with embodiments of the invention.

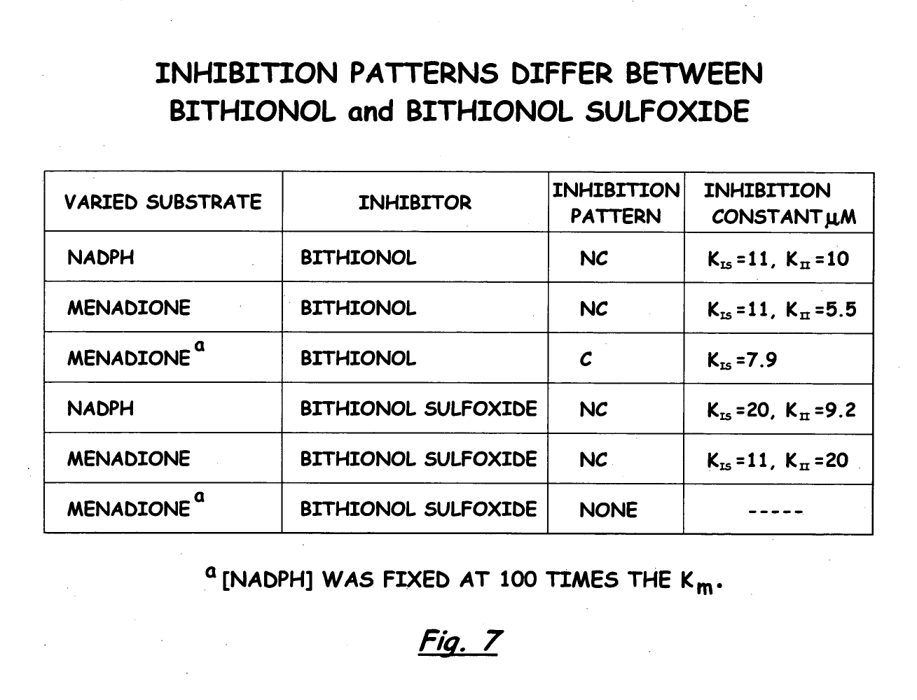

As another approach of estimating possible effective dosages for bithionol and/or bithionol sulfoxide in embodiments of the invention, the inventor has used enzyme inhibition data obtained by his own testing (FIG. 7). The inventor has inserted kinetic constants from Slupe, et al. (Slupe, Williams, Larson, Primbs, Lee, Rogow, Bjorklund, Warner, Peloquin, Shadle, Gambliel, Cusack, Olson, and Charlier, “Reduction of Anthraquinones in 13-deoxydoxorubicin and daunorubicinol by Human Carbonyl Reductase,” Cardiovascular Toxicology, 5, 365-376, 2005) and the data from FIG. 7 of this disclosure into the noncompetitive inhibition equation (equation known in the field of enzyme inhibition), to estimate the concentration of each of bithionol and bithionol sulfoxide required to lower the rate of anthracycline reduction by 50% (which reduction, the inventor believes, would provide a significant, positive effect in improving anthracycline-based cancer treatment). For example, for a clinically relevant concentration of anthracycline (for example, 10 μM of daunorubicin or doxorubicin), the inventor has estimated, using this calculation method, that either 10.5 μM of bithionol, or 11 μM of bithionol sulfoxide, would be sufficient to lower the rate of carbonyl reductase reduction of either 10 μM daunorubicin or 10 μM doxorubicin by 50%. There may be effects caused by the human body, such as low absorption rates in the human stomach, that affect the required dose of bithionol and/or bithionol sulfoxide, but these effects may be determined, and the dosages adjusted, in the course of normal clinical trials. Therefore, the inventor believes that effective and safe doses may be found without undue experimentation by one of skill in the art after reading this disclosure.

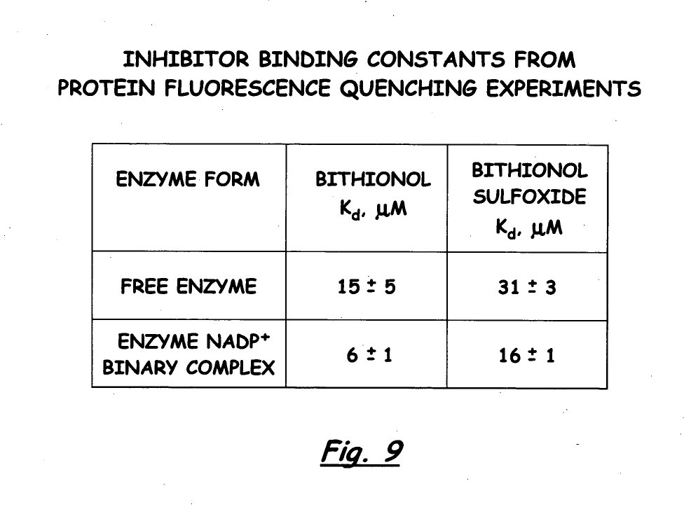

Bithionol and bithionol sulfoxide have been shown by the inventor to be noncompetitive inhibitors against both coenzyme and carbonyl substrates, with Kᵢ values in the low micromolar range (below 10 μM). These preferred compounds have been seen to exhibit inhibition patterns suggestive of binding to multiple enzyme forms, which may mean that increased anthracycline dosages may not overcome the inhibition. Intrinsic protein fluorescence quenching studies have demonstrated that the preferred bithionol and bithionol sulfoxide inhibitors bind to at least the free enzyme and to an enzyme/product binary complex with K_{d} values similar to the Kᵢ values (See FIG. 9), suggesting that inhibition by the compounds is linked to binding to these forms of enzyme.

In use, one or more of the preferred compounds (bithionol, bithionol sulfoxide) may be used in a pharmaceutical composition, which may also comprise one or more of the anthracycline drugs and/or other chemotherapy drugs or other medicines that may be beneficial to the cancer patient. Preferably, the bithionol/bithionol sulfoxide and anthracycline compositions are given at levels that produce the desired anti-cancer effects without the cardiotoxicity side effects. Therefore, the relative compositions may be changed for different anthracyclines and/or for different patients and/or for different cancers. The methods include treatment of, or treatment of side effects, for all cancers for which anthracyclines are used.

### EXAMPLES

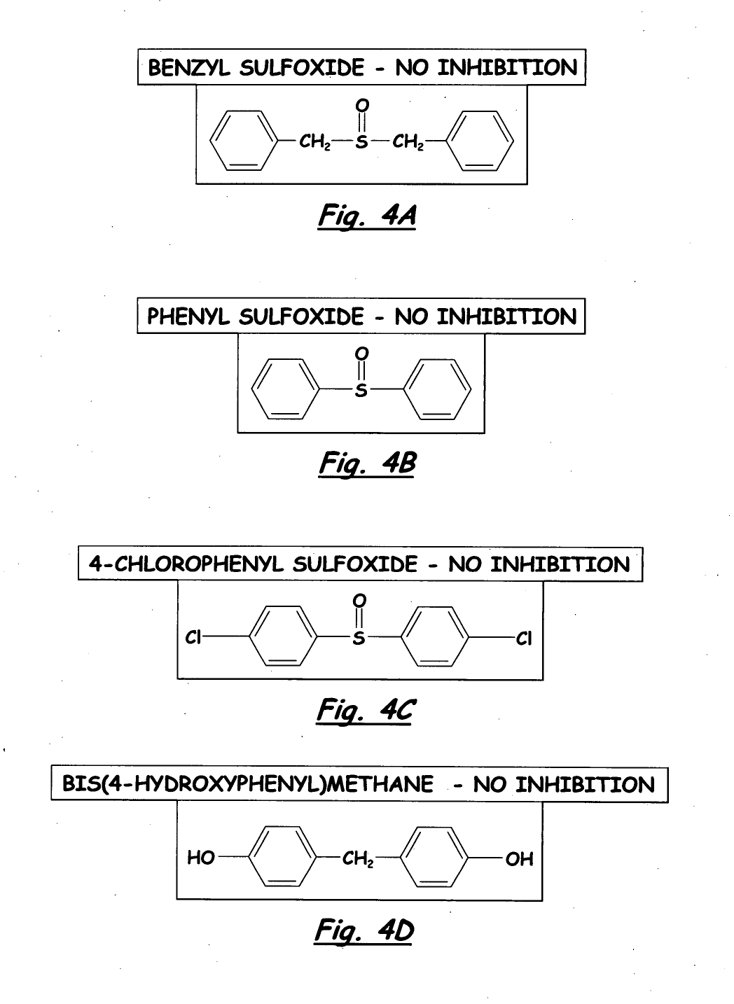

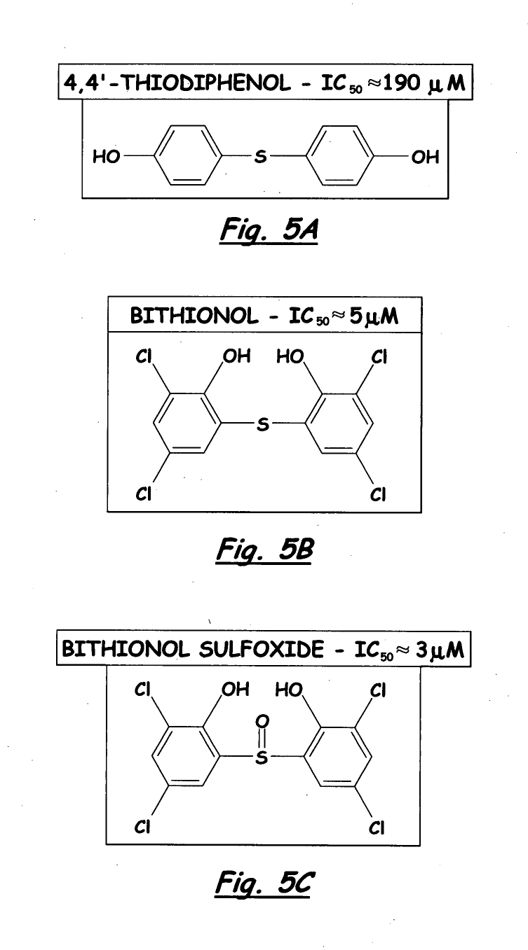

Several analogs of 4-benzoylpyridine were laboratory tested as possible inhibitors for carbonyl reductase, with only three of the analogs tested showing inhibitor behavior. Only two of the inhibitors (bithionol sulfoxide and bithionol) exhibited a low IC₅₀, meaning that only bithionol sulfoxide and bithionol were capable, at low concentrations, in reducing the enzyme activity rate to 50% of that in the absence of the inhibitor. See FIGS. 4A-D for those tested which showed no inhibition (benzyl sulfoxide, phenyl sulfoxide, 4-chlorophenyl sulfoxide, and bis(4-hydroxphenyl)methane), and see FIGS. 5A-C for those that showed at least some inhibition (4,4′-thiodiphenol, bithionol sulfoxide, and bithionol).

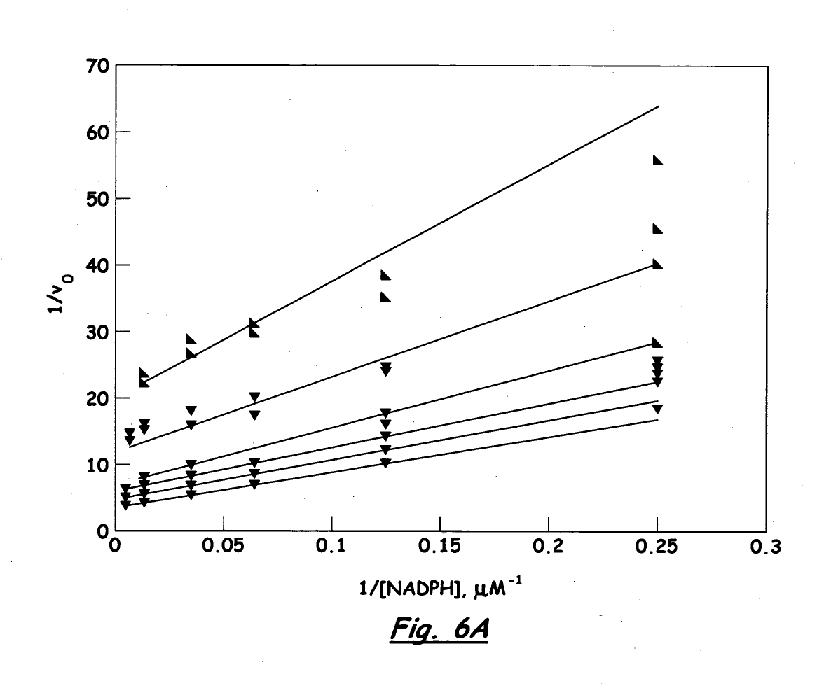

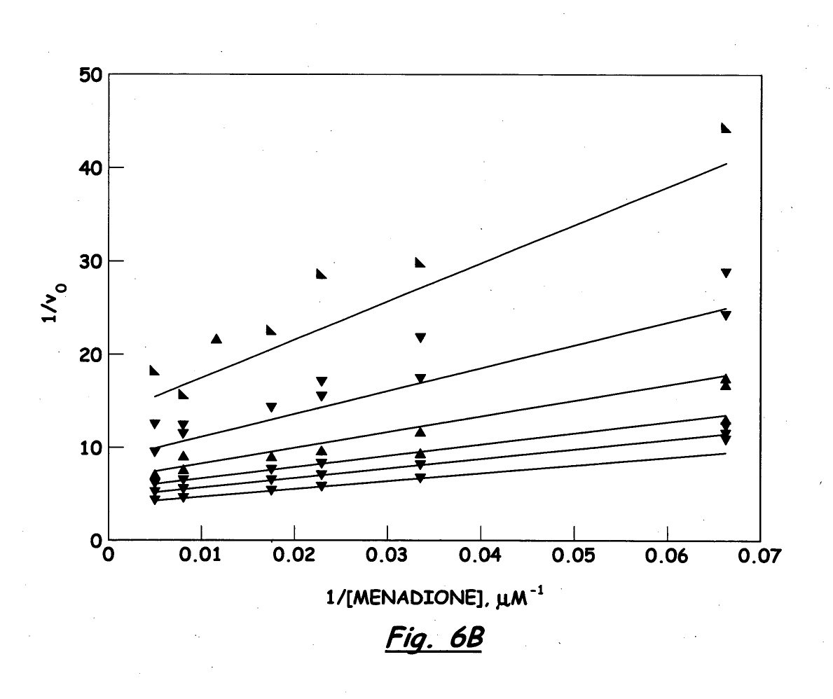

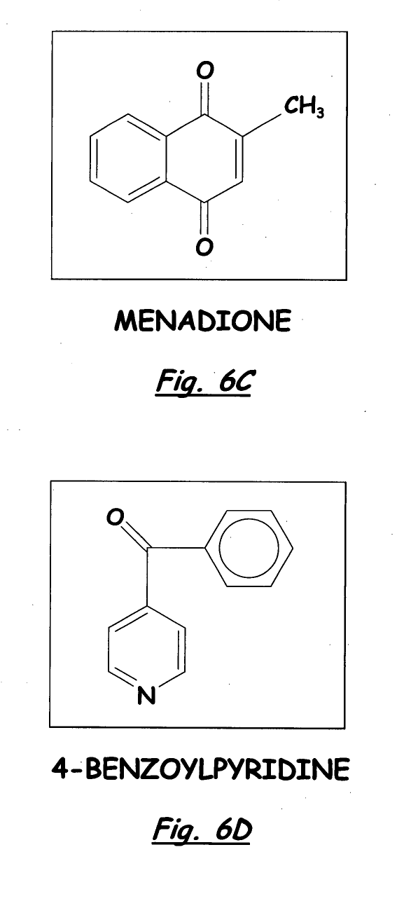

Bithionol sulfoxide was shown, in the inventor's testing, to be a non-competitive inhibitor against both a carbonyl substrate (in these examples, menadione) and NADPH, in tests using subsaturating fixed substrate. See FIGS. 6A and B, portraying 1/rate vs. 1/substrate concentration, with NADPH and menadione as the varied substrates, respectively. As will be understood by those of skill in the art, the FIGS. 6A and B data show changes in both slope and y-intercept, indicating non-competitive inhibition.

In the inventor's testing, inhibition patterns were seen to differ between bithionol and bithionol sulfoxide, as illustrated in FIG. 7.

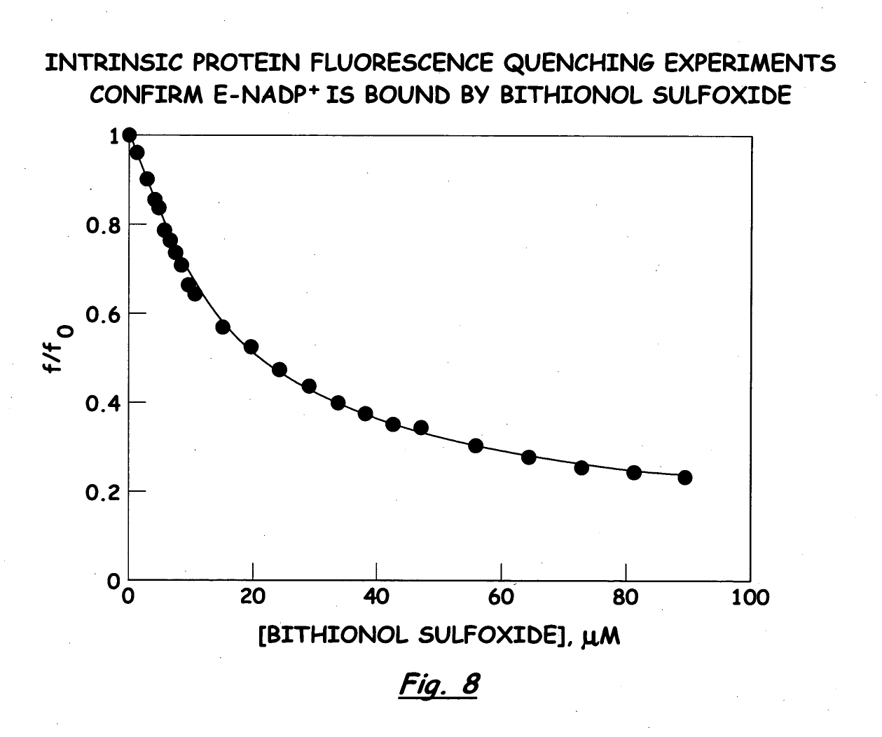

As illustrated in FIGS. 8 and 9, intrinsic protein fluorescence quenching experiments confirmed that bithionol sulfoxide binded to E-NADP⁺, and that both bithionol and bithionol sulfoxide binded to both free enzyme and enzyme NADP⁺ binary complex.

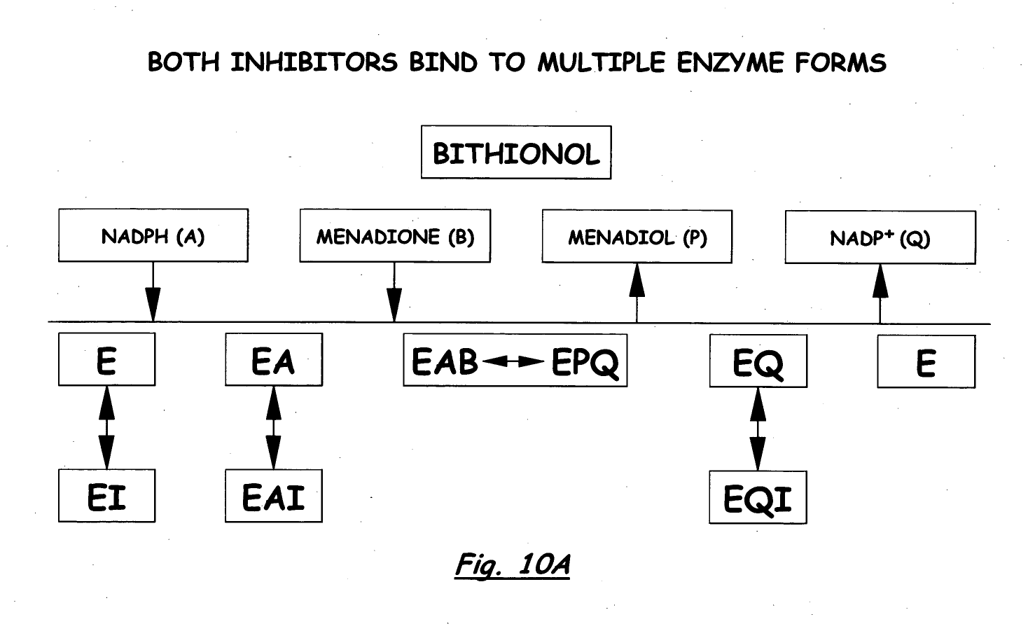

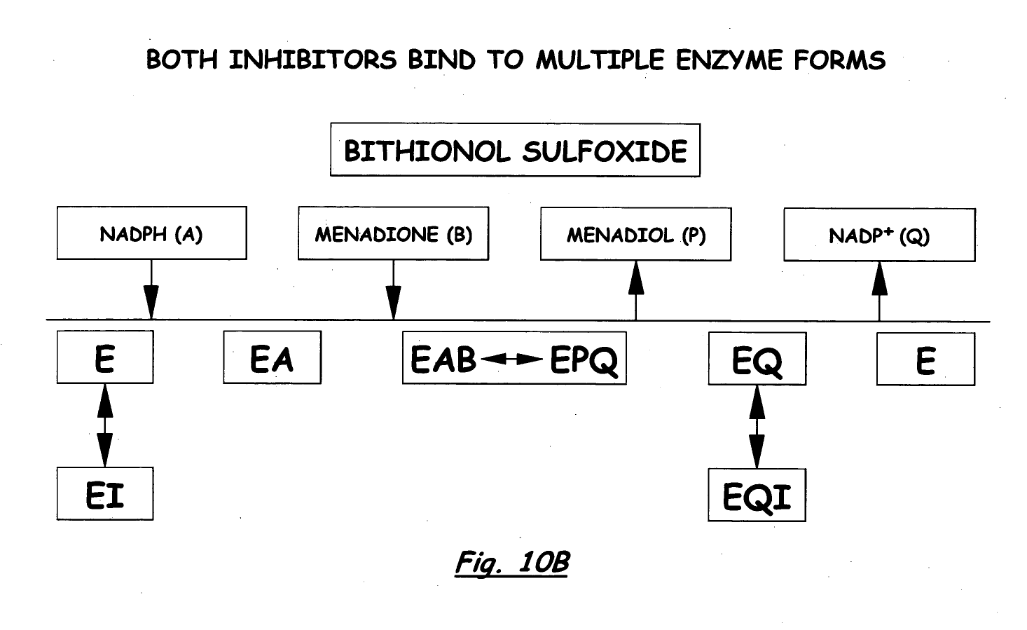

From the above inhibition and protein fluorescence testing, both bithionol and bithionol sulfoxide were seen to bind to multiple enzyme forms, as illustrated in FIG. 10. In FIG. 10, “A” stands for NADPH, “B” stands for the carbonyl compound (menadione in this figure, but may be other carbonyl compounds such as anthracyclines), and “P” stands for menadiol, “Q” stands for NADP⁺, “E” stands for enzyme (carbonyl reductase), and “I” stands for inhibitor (bithionol in FIG. 10A, and bithionol sulfoxide in FIG. 10B). It is expected that substrate carbonyls should not appreciably compete against the preferred inhibitors at these sites. Thus, said preferred inhibitors according to the invention are expected to remain available for, and will effectively carry out, inhibition of the mechanism that would otherwise result in lower efficacy of anthracycline(s) drugs and in cardiotoxic compounds.

Embodiments of the invention therefore include a pharmaceutical composition comprising at least one anthracycline compound and bithionol or bithionol sulfoxide or a mixture thereof. The inventor envisions that there may be analogs or derivatives of bithionol and/or bithionol sulfoxide that also may be effective in compositions and methods of the invention. The compositions may include, for example, anthracycline compounds selected from the group consisting of adriamycin/doxorubicin, daunorubicin/daunomycin, epirubicin, idarubicin, and a mixture of two or more thereof.

While the preferred patients are humans, animals may also benefit from the compositions and methods. Embodiments of the invented method may be for preventing or treating cardiotoxicity associated with anthracycline cancer chemotherapy in a mammal in need thereof, wherein the method comprises administering to the mammal a composition comprising an effective amount of a pharmaceutical composition comprising at least one anthracycline compound and at least one compound or mixture selected from the group consisting of bithionol, bithionol sulfoxide, a mixture of bithionol or bithionol sulfoxide, an analog of bithionol, an analog of bithionol sulfoxide, a derivative of bithionol, a derivative of bithionol sulfoxide, and mixtures thereof. Examples of anthracycline compounds include adriamycin/doxorubicin, daunorubicin/daunomycin, epirubicin, idarubicin, and a mixture of two or more thereof. Effective amounts of said bithionol; bithionol; and mixtures, analogs, derivatives thereof (that is, both amount per weight of the patient and the amount relative to the dose of anthracycline being used) will be determined by methods known to those of skill in the art, using information available from previous clinical use of bithionol in the human body or in animals for liver fluke treatment, and/or from clinical studies using a dose starting point such as may be calculated from laboratory enzyme inhibition data (such as the above-estimated doses calculated by the inventor using data from the testing represented by FIG. 7).

Instead of, or in addition to, administering a pharmaceutical composition including both anthracycline(s) and bithionol and/or bithionol sulfoxide, separate pharmaceutical compositions may be used. For example, methods may include preventing or treating a disease or condition associated with carbonyl reductase in a mammal in need thereof by administering to the mammal a first pharmaceutical composition comprising at least one anthracycline compound; and also administering to the mammal a second pharmaceutical composition comprising bithionol or bithionol sulfoxide, or a mixture thereof. The first and second pharmaceutical compositions may be administered at the same time, or may be administered at nearly the same time (for example, within 15 minutes or less), or preferably within a few hours of each other (for example, within 2 hours or less). It may be beneficial to treat the patient with bithionol and/or bithionol sulfoxide prior to anthracycline therapy (for example, two hours or less prior to anthracycline treatment), to block carbonyl reductase before administration of the anthracycline drug(s).

Thus, it may be said that the preferred composition of bithionol, bithionol sulfoxide, and/or derivatives or analogs thereof, may be administered to a human (or other mammal) in a pharmaceutical composition also comprising at least one anthracycline compound, or may be administered separately from the at least one anthracycline compound either at the same as the anthracycline(s), or any different time found to be effective for inhibiting formation of the anthracycline metabolites.

Although this invention has been described above with reference to particular means, materials, steps, and embodiments, it is to be understood that the invention is not limited to these disclosed particulars, but extends instead to all equivalents within the broad scope of the following claims.

I claim:

1. A pharmaceutical composition comprising at least one anthracycline compound and at least one enzyme inhibitor selected from the group consisting of: bithionol, bithionol sulfoxide, and a mixture thereof.

2. The composition of claim 1 wherein said at least one anthracycline compound is selected from the group consisting of: adriamycin, daunomycin, daunorubicin, doxorubicin, epirubicin, idarubicin, and mixtures of two or more thereof.

3. A method for preventing or treating cardiotoxicity associated with anthracycline cancer chemotherapy in a mammal in need thereof, the method comprising:
administering to the mammal a composition comprising an effective amount of a pharmaceutical composition comprising at least one anthracycline compound and at least one compound or mixture selected from the group consisting of bithionol, bithionol sulfoxide, a mixture of bithionol or bithionol sulfoxide, an analog of bithionol, an analog of bithionol sulfoxide, a derivatives of bithionol, a derivative of bithionol sulfoxide, and mixtures thereof.

4. The method of claim 3 wherein the at least one anthracycline compound is selected from the group consisting of adriamycin, daunomycin, daunorubicin, doxorubicin, epirubicin, idarubicin, and mixtures of one or more thereof.

5. A method for reducing doses of anthracycline effective for cancer chemotherapy in a mammal in need thereof, the method comprising:
administering to the mammal a composition comprising an effective amount of a pharmaceutical composition comprising at least one anthracycline compound and at least one compound or mixture selected from the group consisting of bithionol, bithionol sulfoxide, a mixture of bithionol or bithionol sulfoxide, an analog of bithionol, an analog of bithionol sulfoxide, a derivatives of bithionol, a derivative of bithionol sulfoxide, and mixtures thereof.

6. The method of claim 5 wherein the at least one anthracycline compound is selected from the group consisting of adriamycin, daunomycin, daunorubicin, doxorubicin, epirubicin, idarubicin, and mixtures of one or more thereof.

7. A method for improving efficacy of anthracycline chemotherapy by inhibiting carbonyl reductase in a mammal, the method comprising:
administering to the mammal a first pharmaceutical composition comprising at least one anthracycline compound; and,
also administering to the mammal a second pharmaceutical composition comprising bithionol, bithionol sulfoxide, a mixture of bithionol or bithionol sulfoxide, an analog of bithionol, an analog of bithionol sulfoxide, a derivatives of bithionol, a derivative of bithionol sulfoxide, and mixtures thereof.

8. A method as in claim 7, wherein said first pharmaceutical composition is administered at the same time as said second pharmaceutical composition.

9. A method as in claim 7, wherein said second pharmaceutical composition is administered within two hours or less of said first pharmaceutical composition.

10. A method as in claim 7, wherein said second pharmaceutical is administered prior to the first pharmaceutical composition.

11. A method as in claim 7, wherein said second pharmaceutical is administered after the first pharmaceutical composition.

12. The method of claim 7, wherein said at least one anthracycline compound is selected from a group consisting of adriamycin, daunomycin, daunorubicin, doxorubicin, epirubicin, idarubicin, and a mixture thereof.

13. A method for preventing or treating a disease or condition associated with carbonyl reductase in a mammal in need thereof, comprising the step of:
administering to the mammal a first pharmaceutical composition comprising at least one anthracycline compound; and,
also administering to the mammal a second pharmaceutical composition comprising a compound or mixture selected from the group consisting of bithionol, bithionol sulfoxide, a mixture of bithionol or bithionol sulfoxide, an analog of bithionol, an analog of bithionol sulfoxide, a derivatives of bithionol, a derivative of bithionol sulfoxide, and mixtures thereof.

14. A method as in claim 13, wherein said first pharmaceutical composition is administered at the same time as said second pharmaceutical composition.

15. A method as in claim 13, wherein said second pharmaceutical composition is administered within two hours or less of said first pharmaceutical composition.

16. A method as in claim 13, wherein said second pharmaceutical is administered prior to the first pharmaceutical composition.

17. A method as in claim 13, wherein said second pharmaceutical is administered after the first pharmaceutical composition.

18. The method of claim 13 wherein said anthracycline compound is adriamycin, daunomycin, daunorubicin, doxorubicin, epirubicin, idarubicin, or a mixture thereof.

19. A method for preventing or treating cardiotoxicity associated with anthracycline cancer chemotherapy in a mammal in need thereof, the method comprising:
administering to the mammal a composition comprising an effective amount of a pharmaceutical composition comprising at least one anthracycline compound and at least one compound or mixture selected from the group consisting of bithionol, bithionol sulfoxide, and mixtures thereof.

20. A method for reducing doses of anthracycline effective for cancer chemotherapy in a mammal in need thereof, the method comprising:
administering to the mammal a composition comprising an effective amount of a pharmaceutical composition comprising at least one anthracycline compound and at least one compound or mixture selected from the group consisting of bithionol, bithionol sulfoxide, and mixtures thereof.
